(19) United States
(12) Plant Patent Application Publication
(10) Pub. No.: US 2004/0083515 P1
(43) Pub. Date: Apr. 29, 2004

(54) Chrysanthemum plant named 'Dove Bronze'

(75) Inventor: Keith G. Lintott, Bognor Regis (GB)

Correspondence Address: HOWREY SIMON ARNOLD & WHITE, LLP, BOX 34, 301 RAVENSWOOD AVE., MENLO PARK, CA, 94025, US

(73) Assignee: Cleangro Ltd

(21) Appl. No.: 10/693,365

(22) Filed: Oct. 24, 2003

Related U.S. Application Data
(60) Provisional application No. 60/421,380, filed on Oct. 25, 2002.

(51) Int. Cl. A01H 5/00

(52) U.S. Cl. PLT/289

(57) ABSTRACT

A new variety of Chrysanthemum plant named ‘Dove Bronze’ having a domed canopy of many small single bronze flowers with a very uniform round habit.

This application claims the benefit of U.S. Provisional Application No. 60/421,380, filed Oct. 25, 2002.

### LATIN NAME OF THE GENUS AND SPECIES

Botanical classification: Chrysanthemum morifolium.

### VARIETY DENOMINATION

The new Chrysanthemum variety denomination is ‘Dove Bronze.’

### BACKGROUND OF THE INVENTION

The present invention comprises a new and distinct cultivar of Chrysanthemum botanically known as Chrysanthemum morifolium, and referred to by the cultivar name ‘Dove Bronze.’

‘Dove Bronze’, identified as 30262-1, originated from a whole plant mutation grown in a controlled planting of an unpatented variety commercially available as ‘Dove’ in Chichester, West Sussex PO10 7XQ, United Kingdom. The new variety ‘Dove Bronze’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Dove Bronze’ is a garden type of Chrysanthemum plant having a domed canopy of small single bronze flowers.

### Comparison with Parent

Plants of the new Chrysanthemum variety ‘Dove Bronze’ are similar to the parent ‘Dove’ in habit and growth rate. In side-by-side comparisons in Chichester, West Sussex, United Kingdom under commercial practice, plants of the new Chrysanthemum variety ‘Dove Bronze’ differed from the plants of the parent variety ‘Dove’ in the following characteristic.

1. The new variety ‘Dove Bronze’ produces flowers bronze flowers whereas the parent ‘Dove’ produces pink flowers.

### Comparison with Other Varieties

Plants of the new Chrysanthemum variety ‘Dove Bronze’ are dissimilar to the variety ‘Grace’ (U.S. Plant Pat. No. 7,642) in plant habit and growth. In side-by-side comparisons in Chichester, West Sussex, United Kingdom, under commercial practice, plants of the new Chrysanthemum variety ‘Dove Bronze’ differed from ‘Grace’ in the following characteristics.

1. The new variety ‘Dove Bronze’ produces smaller sized flowers than ‘Grace.’

2. The new variety ‘Dove Bronze’ usually produces more flowers than ‘Grace.’

3. The new variety ‘Dove Bronze’ has a more round and domed shape than ‘Grace.’

### BRIEF DESCRIPTION OF ILLUSTRATION

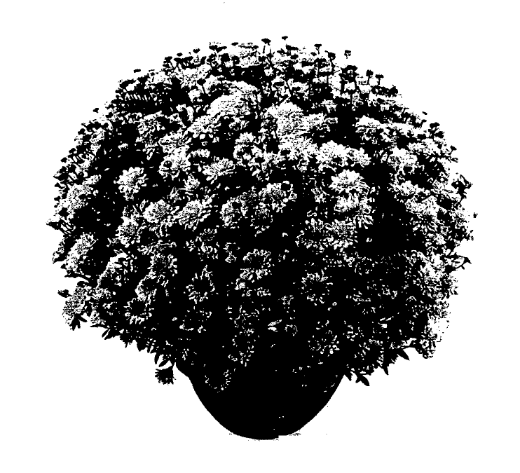

Typical specimens of the plant and flowers for the new Chrysanthemum variety ‘Dove Bronze’ are shown in the accompanying digital photograph. The colors shown are as true as possible within the usual limits of this kind of illustration. FIG. 1 is a whole plant view of the new variety ‘Dove Bronze’ grown in a pot. The plant shown in the illustration is 63 days from commencement of planting outdoors in mid-June.

### DETAILED BOTANICAL DESCRIPTION

The following description of the new Chrysanthemum variety ‘Dove Bronze’ is of plants grown outside, natural season in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 50° F. at night and about 70° F. to 75° F. during the day under natural light conditions. The plants were grown outdoors in mid-June in 4 liter pots using loamless compost. No growth retardants were used. To produce a commercial product the plants may be pinched once with the center bud removed. The typical container size for commercial growth is 4 liters (8″). It has been observed that the shelf life of the new variety is about 25 days with a response time of about 9 weeks. The new variety is suitable for growth in a temperature range of 50° F. to 75° F. The new variety may be produced as a pot plant. The following description is with respect to a plant produced as a garden pot plant. In the description of this new Chrysanthemum variety, color values have been taken from The Royal Horticultural Society Colour Chart (R.H.S.C.C.).

### Plant

Plant type: Pot.

Habit: Domed — Round.

Height: 40 cm.

Width: 50 cm.

Branching characteristics: Angular and Free Branching.

Length of lateral branches: 14-32 cm.

Number of breaks from pinch: 7-9.

Stem color: near 137A.

Response time: 63 days natural season.

Vigor: Medium.

Shelf life: 25 days.

Disease (susceptibility/resistance observed): None observed to date.

Pest (susceptibility/resistance observed): None observed to date.

Growth retardant type and treatment: No treatment necessary.

The plants were grown for 10 weeks in natural season conditions.

Propagation:

Type.—Vegetative Propagation via Stem Cuttings.

Time to rooting.—12 days with soil temperatures of 70° F.

Rooting habit.—Fine and Fibrous.

### Foliage

Number of leaves per lateral branch: 10-15.

Compound or single: Single.

Arrangement of leaves: Alternate.

Shape of leaf.—typically 5 lobed ovate.

Size of leaf.—Width (cm): 1-4 Length (cm): 2-5.5.

Leaf apex.—Acute.

Base.—Truncate.

Attachment.—Petioled.

Aspect.—Slightly Undulating.

Margin.—Palmately Lobed.

Surface characteristics.—Top: Very Slightly Pubescent. Bottom: Very Slightly Pubescent.

Petiole:

Color.—near 139C.

Length: 0.8 cm-1.2 cm.

Venation: Net prominent mid-vein at underside.

Color.—upper side: near 138D. underside: near 138D.

Color:

Mature leaf, upper side.—near 137C; under side: near 137D.

Young leaf, upper side.—near 138B; under side: near 138B.

### Flower

Flower appearance: Matte.

Flower type: Single.

Flower form: Flat.

Flower shape: Round.

Flowering habit: Cyme.

Number of blossoms per branch: 8-12.

Inflorescence form: Corymibiform.

Depth of fully expanded blossoms.—1 cm.

Diameter of fully expanded blossoms.—2-3.5 cm.

Phyllaries:

Number.—10-12.

Color.—near 137C.

Length.—4-8 mm.

Width.—1-2 mm.

Texture/Appearance.—Very Slightly Pubescent.

Pedicel:

Length.—terminal: 4 cm; lateral: 7-8 cm.

Color.—near 136C.

Surface.—Pubescent.

Habit.—Slightly curving.

Strength.—Medium.

Ray florets:

Form/Shape.—Straight.

Texture/Appearance.—Matte.

Number per flower.—35-38.

Length.—1-1.5 cm Width.—3-5 mm.

Apex.—Rounded, Slightly Dentate.

Base.—Tubular.

Margin.—Entire.

Disc florets:

Form/Shape.—Cylindrical.

Texture/Appearance.—Shiny.

Number per flower.—125-140.

Length.—1-1.5 cm; Width: 1 mm.

Diameter of disc.—6-8 mm.

Fragrance: None.

Flower bud (at onset of color):

Length.—6 mm.

Diameter.—7 mm.

Form/Shape.—Globular.

General flower color:

1. Ray florets, upper side.—Immature: near 171B. Mature: near 171C. Older/Fading: near 164C.

2. Ray florets, under side.—Immature: near 171B. Mature: near 171C. Older/Fading: near 164C.

3. Disc florets.—Immature: near 17B. Mature: near 16B. Older/Fading: near 16C.

4. Bud.—near 145C.

Flower progression with age: Color fades uniformly with age.

### Reproductive Organs

Gynoecium present on ray and disc florets:

Ray florets per individual flower:

Pistil number.—35-38.

Stigma color.—near 144D.

Stigma shape.—Forked.

Style color.—near 144D.

Style length.—4 mm.

Disc florets per individual flower:

Pistil number.—100-110.

Stigma color.—near 150C.

Stigma shape.—Cylindrical.

Style color.—near 150C.

Style length.—2-3 mm.

Androecium present on disc florets only:

Stamen number.—25-40.

Anther shape.—Cylindrical.

Anther color.—near 14C.

Pollen color.—near 12A.

Pollen quantity.—Abundant.

Fruit and seeds: None observed.

What is claimed is:

1. A new and distinct variety of Chrysanthemum plant, substantially as described and illustrated herein.
